(19) United States
(12) Patent Application Publication
(10) Pub. No.: US 2016/0346340 A1
(43) Pub. Date: Dec. 1, 2016

(54) COMPOSITIONS INCLUDING MILK THISTLE AND METHODS FOR THE TREATMENT OF VARIOUS DISORDERS USING THE SAME

(71) Applicant: John E. Poulos, Fayetteville, NC (US)

(72) Inventor: John E. Poulos, Fayetteville, NC (US)

(21) Appl. No.: 15/139,740

(22) Filed: Apr. 27, 2016

Related U.S. Application Data
(60) Provisional application No. 62/166,227, filed on May 26, 2015.

(51) Int. Cl. A61K 36/28; A61K 31/7076; A61K 31/522; A61K 31/714; A61K 31/197; A61K 31/355

(52) U.S. Cl. CPC A61K 36/28; A61K 31/197; A61K 31/355; A61K 31/522; A61K 31/714; A61K 31/7076

(57) ABSTRACT

The present invention provides compositions including milk thistle or a constituent thereof for the treatment of disorders including metabolic syndrome, metabolic disorders, diabetes-related disorders, cardiovascular disorders or liver disease.

### RELATED APPLICATION DATA

This application claims the benefit of U.S. Provisional Application No. 62/166,227 filed May 26, 2015, The entirety of the content of this application is hereby incorporated by reference for all purposes.

### FIELD OF THE INVENTION

The present invention relates to compositions including milk thistle and methods for treating metabolic disorders, metabolic syndrome, diabetes-related disorders, cardiovascular disorders or liver disease using the same.

### BACKGROUND OF THE INVENTION

According to 2009 Center for Disease Control (CDC) data, approximately one-third of Americans are obese, and over the past decade there has been an approximate two-fold increase in the prevalence of obesity (Centers for Disease Control and Prevention. U.S. Obesity Trends—Trends by State 1985-2008. Aug. 19, 2009. Sep. 11, 2009. Ref Type: Online Source). Obesity is associated with the metabolic syndrome and is estimated to affect 47 million Americans, The diagnosis of the metabolic syndrome is based on three or more of the following: Abdominal obesity (waist>40″ for men and 34.5″ for women), triglyceride level>150 mg/dL, HDL<40 mg/dL for men and <50 mg/dL for women, fasting blood glucose ≧110 mg/dL, and blood pressure≧130/85 (Grundy S M, Cleeman J I, Daniels S R, et al. Diagnosis and management of the metabolic syndrome: an American Heart Association/National Heart, Lung, and Blood Institute

Scientific Statement, Circulation 2005;112:2735-2752). Non-alcoholic fatty liver disease (NAFLD) is strongly associated with the metabolic syndrome and obesity and its presence as the number one cause of chronic liver disease correlates with increasing rates of obesity and the metabolic syndrome (Kida Y, Sato T. The metabolic syndrome as a predictor of nonalcoholic fatty liver disease. Ann Intern Med 2006;144:379-380; Farrell G C, Larter C Z. Nonalcoholic fatty liver disease: From steatosis to cirrhosis. Hepatology 2006;43:S99-112). NAFLD is characterized by high levels of fat or triglycerides in the liver with or without inflammation and scarring. NAFLD may also encompass steatohepatitis (NASH) and progress to cirrhosis of the liver. NAFLD is rapidly becoming the leading cause of liver disease in the US seen in 20% of obese individuals and possibly affecting 20 million Americans (Adams L A, Lymp F J, T S Sauver et al. The natural history of nonalcoholic fatty liver disease; a population-based cohort study. Gastroenterology 2005;129:113-121; Browning J Szczepaniak L , Dobbins R et al, Prevalence of hepatic steatosis in an urban population in the United States: impact of ethnicity. Hepatology 2000;40:1387-1395). Thirty to forty percent of patients with NAFLD will have hepatic scarring and of this, 10-15% will develop cirrhosis of the liver (Ekstedt M, Franzen L E, Mathiesen U L et al. Long-term follow-up of patients with NAFLD and elevated liver enzymes. Hepatology 2006; 44;865-73; Matteotti C A, Younossi Z M, Gramlich T. Nonalcoholic fatty liver disease: a spectrum of clinical and pathological severity. Gastroenterology 1999;116:1413-1419).

NAFLD is rapidly becoming a major health issue due to its association with the metabolic syndrome, type two diabetes mellitus and cardiovascular disease (Fabbrini, E, Sullivan S and Klein S. Obesity and Nonalcoholic Fatty Liver Disease: biochemical, metabolic and clinical implications. Hepatology 2010;51:6867-6889). Recent studies have shown that NAFLD is associated with an increased prevalence of cardiovascular disease and increased risk of cardiac death (Pacifico L, Cantisani V, Ricci P. Nonalcoholic fatty liver disease and carotid arterosclerosis in children. Pediatr Res 2008;63:423-427; Adams L A, Lymp J F, St Sauver J. The natural history of nonalcoholic fatty liver disease: a population-based cohort study. Gastroenterology 2005;129:113-21). In fact, the most common cause of death in NAFLD patients is not from liver-related causes but from coronary artery disease (Ong J P, Pitts A, Younossi Z M. Non-alcoholic fatty liver disease (NAFLD) is associated with higher overall mortality and liver related mortality. Journal of Hepatology 2008;48:S5-S5). The presence of NAFLD may also be a predictor for the future development of metabolic syndrome and diabetes. Glucose intolerance and insulin resistance has also been thund to occur in the early states of chronic liver disease and subjects with NAFLD are three times more likely to develop diabetes and 50% more likely to develop the metabolic syndrome than the general population (Adams L A, Waters O R, Knuiman M W et al., NAFLD as a Risk Factor for the Development of Diabetes and the Metabolic Syndrome: An Eleven-Year Follow-up Study Am J Gastroenterol 2009;104:861-86; Buzzeili G, Chiarantini F, Cotrozzi G. Estimate of prevalence of glucose intolerance in chronic liver disease. Degree of agreement among some diagnostic criteria. Liver 1988;8:354-359). There is also increasing evidence that patients with NAFLD or elevated hepatic enzymes indicative of NAFLD without known diabetes should be screened for diabetes (Ghouri, N, Preiss D, and Sattar N Liver Enzymes, Nonalcoholic Fatty Liver Disease, and Incident Cardiovascular Disease: A Narrative Review and Clinical Perspective of Prospective Data. Hepatology 2010; 52:1156-1161). Abnormalities in liver function tests are associated with hepatic fat and have been shown to correlate with a higher hazard ratio for the development of diabetes (Fraser A, Harris R, Sattar N. Alanine aminotransferase, gamma-glutamyltransferase, and incident diabetes: the British Women's Heart and Health Study and meta-analysis. Diabetes Care 2009;32:741-750). Diabetes and obesity have also been associated with the development of liver cancer (Verna E C, Berk P D. Role of fatty acids in the pathogenesis of obesity and fatty liver: impact of bariatric surgery. Sevin Liver Dis 2008;28:407-426). There maybe common pathophysiologic mechanisms underlying the development of type 2 diabetes, cardiovascular disease and NAFLD.

### SUMMARY OF THE INVENTION

Embodiments of the present invention are directed to compositions including a combination of any of the following active agents: milk thistle or a constituent thereof, vitamin E, carnitine, caffeine, vitamin B₁₂, and S-adenosylmethionine (SAMe).

Embodiments of the present invention provide a composition including milk thistle or a constituent thereof; vitamin E; carnitine; caffeine; and a physiologically acceptable carrier.

Embodiments of the present invention provide a composition including milk thistle or a constituent thereof; vitamin E; carnitine; vitamin B₁₂; and a physiologically acceptable carrier.

Embodiments of the present invention provide a composition including milk thistle or a constituent thereof; vitamin E; carnitine; caffeine; vitamin B₁₂; and a physiologically acceptable carrier.

Embodiments of the present invention also provide methods of preventing or treating metabolic syndrome, a metabolic disorder, a diabetes-related disorder, a cardiovascular disorder or liver disease comprising administering to a subject a composition of the present invention.

Embodiments of the present invention are also directed to methods of preventing or treating metabolic syndrome, a metabolic disorder, a diabetes-related disorder, a cardiovascular disorder or liver disease comprising administering to a subject a composition including milk thistle or a constituent thereof, vitamin E, carnitine and a physiologically acceptable carrier, wherein the diabetes-related disorder or the liver disease is not fatty liver disease or non-alcoholic fatty liver disease (NAFLD).

Embodiments of the present invention also provide methods of preventing or treating metabolic syndrome including administering to a subject a composition including milk thistle or a constituent thereof, vitamin E carnitine and a physiologically acceptable carrier or a composition including a combination of any of the following active agents: milk thistle or a constituent thereof, vitamin E, carnitine, caffeine, vitamin B₁₂, and S-adenosylmethionine (SAMe).

### DETAILED DESCRIPTION

The present invention is further described below in greater detail. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

All patent and patent application references referred to in this patent application are hereby incorporated by reference in their entirety as if set forth fully herein.

A. Definitions

As used herein, “a,” “an” or “the” can mean one or more than one. Also as used herein, “and/of” refers to and encompasses any and all possible combinations of one or more of the associated listed items, as well as the lack of combinations when interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” as used herein should not be interpreted as equivalent to “comprising.”

The term “about,” as used herein when referring to a measurable value such as an amount of dose (e.g., an amount of a compound) and the like, is meant to encompass variations of ±20%, ±10%, ±5%, ±1%, ±0.5%, or even ±0.1% of the specified amount.

As used herein, a “metabolic disease” or “metabolic disorder” (wherein disease and disorder can be used interchangeably) refers to a condition caused by an abnormal metabolic process. Common metabolic disorders include, but are not limited to, diabetes, insulin resistance, obesity, dyslipidemia, hyperlipedemia, hyperthyroidism, hypothyroidism, galactosentia and pherlyiketonuria.

The term “metabolic syndrome” refers to a cluster of conditions including increased blood pressure, a high blood sugar level, excess body fat around the waist and abnormal cholesterol levels that occurring together can increasing the risk of heart disease, stroke and/or diabetes. “Metabolic syndrome” further includes a disease diagnosed as metabolic syndrome according to the diagnostic standard, for example, by the WHO, NCEP, IDF or the Committee for Diagnostic Standard of Metabolic Syndrome in the Japan Atherosclerosis Society.

“Diabetes” can refer to a disease diagnosed as diabetes according to the diagnostic standard, for example, of WHO (World Health Organization), Japan Diabetes Society, American Diabetes Association or European Association for the Study of Diabetes and includes Type 1 diabetes, Type 2 diabetes, gestational or pregnancy diabetes, and the like. Type 2 diabetes can be characterized by its resistance to the action of insulin, i.e., “insulin resistance.” “Insulin resistance” can mean a disease diagnosed as insulin resistance, based on the insulin resistance index (fasting blood sugar (mg/dL)×fasting insulin (microU/mL)÷405) or on the results obtained by examination by glucose clamp method or the like and includes syndrome X additionally. In addition to Type 2 diabetes, diseases with “insulin resistance” include, for example, fatty Liver, particularly non-alcoholic fatty liver disease (NAFLD), non-alcoholic steatohepatitis (NASH), coronary heart diseases (CHDs), arteriosclerotic diseases, hyperglycemia, lipodosis, impaired glucose tolerance, hypertension, hyperlipemia, diabetes complications, pregnancy diabetes, polycystic ovary syndrome and the like.

The term “dyslipidemia” means a disease diagnosed as dyslipidemia according to the diagnostic standard, for example, by the WHO or Japan Atherosclerosis Society and includes hyperlipemia, hypercholestrolemia, hyper-LDL-cholestrolemia, hypo-HDL-cholestrolemia, hypertriglyceridemia and the like.

“Obesity” as used herein refers to a state of excess body fat mass. Obesity can include both excess body weight and excess adipose tissue mass in a subject. An obese subject may include a subject having a body mass index of about 30 kg/m². An obese subject may further include a subject whose body weight is twenty percent (20%) or more above the level considered by the U.S. Public Health Department, and/or similar organizations to be normal or healthy for the subject's age and gender. According to some standards, obesity is defined as a body mass index (BMI) equal to or more than about 30. See Center for Disease Control (CDC) standards for obesity. According to additional standards, generally, the normal amount of body fat (expressed as percentage of body fat) is between about 25-30% in women and about 18-23% in men. Women with over about 30% body fat and men with over about 25% body fat are considered obese.

“Overweight” as used herein refers to a state wherein a subject has a body weight greater than the level considered by the U.S. Public Health Department, and/or similar organizations to be normal or healthy for the subject's age and gender. According to some standards, overweight is defined as a BMI equal to or more than about 25 where normal weight is defined as a BMI of about 18.5 to 24.0. According to some standards an adult who has a BMI between 25 and 29.9 is considered overweight. See Center for Disease Control (CDC) overweight definition.

Methods of estimating body fat and body fat distribution further include measurements of skinfold thickness and waist circumference, calculation of waist-to-hip circumference ratios, and techniques such as ultrasound, computed tomography, and magnetic resonance imaging (MRI).

However, in any circumstance, a diagnosis of any disease or disorder described herein may be made by clinical observation and assessment and/or through diagnostic testing recognized as acceptable by those skilled in the art for determining the amount and/or duration of therapy.

“Physiologically acceptable carrier” and “pharmaceutically acceptable carrier” which may be interchangeably used refer to a carrier that does not cause significant irritation to an organism and does not abrogate the biological activity and properties of the administered compound.

A “carrier” refers to, for example, a diluent adjuvant, excipient, auxilliary agent or vehicle with which an active agent of the present invention is administered and can include sterile water, saline, glucose, dextrose, stabilizers (e.g., sugars and amino acids), preservatives, wetting agents, emulsifying agents, pH buffering agents, additives that enhance viscosity, and the like. Suitable carriers are described in “Remington's Pharmaceutical Sciences” by E. W. Martin.

“Effective amount” as used herein refers to an amount of an agent, composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

By the term “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder.

A “treatment effective” amount as used herein is an amount that is sufficient to treat (as defined herein) the subject. Those skilled in the art will appreciate that the therapeutic effects need not be complete or curative, as long as some benefit is provided to the subject.

The term “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the term “prevent,” “preventing,” or “prevention of” (and grammatical variations thereof) refer to prevention and/or delay of the onset and/or progression of a metabolic disease in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

“Subjects” as used herein are generally human subjects and include, but are not limited to, “patients.” The subjects may be male or female and may be of any race or ethnicity, including, but not limited to, Caucasian, African-American, African, Asian, Hispanic, Indian, etc. The subjects may be of any age, including newborn, neonate, infant, child, juvenile, adolescent, adult, and geriatric. Subjects may also include animal subjects, particularly mammalian subjects such as canines, felines, bovines, caprines, equines, ovines, porcines, rodents (e.g. rats and mice), lagomorphs, primates (including non-human primates), etc., for weight control purposes as well as veterinary medicine and/or pharmaceutical drug development purposes. Subjects further include, but are not limited to, those who are of normal weight or are afflicted with or at risk for being overweight or obese. Risk factors for obesity include, but are not limited to, poor diet, lack of physical activity, working varied shifts, certain medications, rare hereditary diseases, and hormonal imbalances (such as hypothyroid and Cushing's disease), cessation of smoking, increased age, genetic factors, race and cultural influences.

A subject of this invention can include an animal subject, particularly mammalian subjects such as canines, felines, bovines, caprines, equines, ovines, porcines, rodents (e.g. rats and mice), lagomorphs, primates (including non-human primates), etc., for veterinary medicine or pharmaceutical drug development purposes.

B. Active Agents

Active agents of the compositions of the present invention include combinations of the following: milk thistle or a constituent thereof, vitamin E, carnitine, caffeine, vitamin B₂, and S-adenosylmethionine (SAMe). in some embodiments, the compositions comprise, consist or consist essentially of a combination of the active agents recited above and herein along with a physiologically acceptable carrier. The active agents are described in greater detail below.

Milk Thistle.

Milk thistle (Silybum marianum), which is also commonly known as Marian thistle, St. Mary's thistle, and Our Lady's thistle, is a native to the Mediterranean region, but has been naturalized in California and the eastern United States. This tall herb with prickly variegated leaves and milky sap has been used as a folk remedy for liver and biliary complaints for many years and recent research has supported such medicinal use (Foster, S., A Field Guide to Medicinal Plants, Houghton Mifflin Co, Boston, 1990, p. 198). Research over the past 20 years has documented that the plant contains a compound referred to as silymarin, which actually includes various forms o hepatoprotectant flavonotignans, The principal components are silybin (which is also called silybinin); silychristin; and silydianin (which is also called silyinonin); the 3-deoxy-derivatives of silychristin and silydianin; as well as b3osilychristin; isosilybin (which is also known as isosilybinin) and its 3-deoxy derivative silandrin; the 3-deoxy compounds silyhermin A and B; 2,3 dehydrosilybin; and the trimers, quatramers and pentamers of silybin (which collectively are referred to as silybinomers). Other flavanolignans may he included as well. Isomers of silybin (or silybinin) are silybin A and B (or silybinin A and B). Milk thistle is often available as an extract that contains silymarin, but it is envisioned that any form or formulation of milk thistle, e.g., extract, precipitate, or powdered form, which contains either silymarin or one or more active components of silymarin, would function in the present invention. Silibinin is the major constituent of silymarin (in a 50:50 mixture of Silybin A and Silybin B) Further constituents include isosilibinin (isosilybin A and isosilybin B), silidianin (silydianin), silichristin (silychristin), isosilychristin, taxifolin and others. Methods for isolating silibinin are known from the prior art (e.g., U.S. Pat. No. 4,871,763).

Vitamin E.

Vitamin E is now considered a generic name describing bioactivities of both tocopherols and tocotrienols derivatives, and vitamin E is the general name for a class of eight compounds: four isomers of tocopherol (alpha-tocopheral, beta-tocopherol, gamma-tocopherol, delta-tocopherol) and four isomers of tocotrienol (alpha-tocotrienol, beta-tocotrienol, gamma-tocotrienol, delta-tocotrienol). Structurally, tocopherols and tocotrienois share some resemblance consisting of common chromanol head and side chain at the C-2 position. Tocopherols and tocotrienols are sometimes collectively called tocols.

Vitamin E is a fat soluble vitamin necessary in the diet of many species for normal reproduction, normal development of muscles, resistance of erythrocytes to hernolysis and various biochemical functions. The most broadly acknowledged function of vitamin E, whereby it is an antioxidant. The vitamin E content in crude palm oil ranges between 600-1000 parts per million (ppm) and is a mixture of tocopherols (18-22%) and tocotrienols (78-82%).

Carnitine.

Carnitines are also called vitamin B_{T}, which are biosynthesized from essential amino acids: lysine and methionine in liver/kidney. Carnitines can penetrate a cell membrane with comparative ease and thus they are distributed to each organ and also excreted to urine. Well known carnitines are. L-carnitine, carnitine chloride, levocarnitine chloride, acetylcarnitine, etc. Especially, levocarnitine chloride is on sale (commercial name: L-Cartin tablets), which has an efficacy for improving levocarnitine deficiency in propionic academia or methylmalonic acidemia, and also it is indicated that levocarnitine chloride may be useful for improving circulatory failure in lower limbs. Further, L-carnitine is also known as a food material.

Carnitine includes L-carnitine, carnitine chloride, levocarnitine chloride, acetylcarnitine and the like, especially L-camitine and levocarnitine chloride are preferable.

Caffeine.

Caffeine is a bitter, white crystalline xanthine alkaloid. It is quickly absorbed through the gastrointestinal tract. It is then metabolized by the liver and through enzymatic action results in three metabolites: paraxanthine, theophylline, and theobromine. it has been shown that elevated levels can appear in the bloodstream within 15 to 45 minutes of consumption, and peak concentrations are evident one hour post ingestion. Circulating concentrations are decreased by 50 to 75% within three to six hours of consumption. Caffeine crosses the membranes of nerve cells as well as muscle cells and it has been proposed that its effects may he more neural than muscular. It has also been proposed that caffeine may have more powerful effects at steps other than metabolism in the process of exciting and contracting the muscle.

Vitamin B₁₂.

Vitamin B12 or “B12” refers to a family of substances composed of tetrapyrrole rings surrounding a central cobalt atom with nucleotide side chains attached to the cobalt. The overall group name is cobalamin, with each of the different cobalt-linked upper axial ligands conferring a different name: methyl (methylcobalamin), hydroxyl (hydrocobalamin) H₂O (aquacobalamin), cyanide (cyanocobalamin) and 5-deoxyadenosine (deoxyadenosylcobalamin) (Klee, G., Clin, Chern. 46(8B):1277-1283 (2000) at page 1277).

Vitamin B12 is also referred to as cyanocobalamin or cobalamin. Vitamin B12 derivatives include in particular cobalamins in which the cyano group of the cyanocobalamin is replaced by other cobalt coordination partners. These include in particular hydroxocobalamin, aquocobalamin, nitrosocobalamin, methylcobalamin and adenosylcobalamin (coenzyme B12).

Physiologically acceptable salts of vitamin B12 or vitamin B12 derivatives include in particular acid addition salts, e.g. with the abovementioned inorganic and organic acids. The acetate of hydroxocobalamin should be mentioned in particular.

S-adenosylmethionine.

S-adenosylmethionine (SAMe) is a physiologic compound that is present throughout body tissue and takes part in a number of biologic reactions as a methyl group donor or an enzymatic activator during the synthesis and metabolism of hormones, neurotransmitters, nucleic acids, phospholipids, and proteins. It is naturally formed in the body from ATP and methionine. SAMe is involved in many biochemical reactions including transmethylation, transsulfation, and synthesis of amines (Stramentinoli, G., Pharmacologic Aspects of S-Adenosylmethionine, American Journal of Medicine 83 (5A), 1987, pp, 35-42). In higher organisms, SAMe plays a significant role in transmethylation processes in more than 40 anabolic or catabolic reactions involving the transfer of the methyl group of SAMe to substrates such as nucleic acids, proteins and lipids, among others. SAMe additionally has anti-oxidant effects via its derivatives (e.g., methylthioadenosine), which prevent oxidative damage to ells. Glutathione itself is a product of SAMe via the transmethylation and transsuifation pathways.

C. Formulations

The active agents described herein may be formulated for administration in a physiological carrier in accordance with known techniques. See, e.g., Remington, The Science and Practice of Pharmacy (latest edition). The particular choice of carrier and formulation will depend upon the particular route of administration for which the composition is intended.

The compositions of the present invention may be suitable for parenteral, oral, inhalation spray, topical, rectal, nasal, buccal, vaginal or implanted reservoir administration, etc. The term “parenteral” as used herein includes subcutaneous, intravenous, intramuscular, intra-articular, intra-synovial, intrastemal, intrathecal, intrahepatic, intralesional and intracranial injection or infusion techniques. in particular embodiments, the compounds are administered orally.

For oral administration, the active compounds may be provided in an acceptable oral dosage form, including, but not limited to, capsules, tablets, aqueous suspensions or solutions. In the case of tablets for oral use, carriers commonly used include lactose and corn starch. Lubricating agents, such as magnesium stearate, may also be added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Formulations suitable for buccal (sub-lingual) administration include lozenges or gels comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound(s), which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a nontoxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions, These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The formulations may be presented in unit\dose or multi-dose containers. for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s), or a salt thereof, in a unit dosage form in a sealed container. The compound or salt is provided in the form of a lyophilizate which is capable of being reconstituted with a suitable physiologically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6)318 (1986)) and typically take the form of an optionally buffered aqueous solution of the active compound. Suitable formulations comprise citrate or his\tris buffer (pH 6) or ethanol/water and contain from 0.1 to 0.2 M active ingredient.

In addition to active agents, the compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

The amount of the active agents in the compositions that may be combined with the carrier materials to produce a composition in a single dosage form will vary depending upon the subject and condition treated and the particular route of administration.

In one embodiment, for oral dosing, suitable daily dosages are, for example, between about 0.1-5000 mg administered (including all numerical values in between) orally once-daily, twice-daily, or three times-daily, continuous (every day) or intermittently (e.g., 3-5 days a week).

D. Methods

The present invention provides methods of preventing or treating metabolic syndrome, metabolic disorders, diabetes-related disorders, cardiovascular disorders and/or liver disease including administering to a subject a composition of the present invention.

Embodiments of the present invention, also provide methods of preventing or treating metabolic syndrome, metabolic disorders, diabetes-related disorders, cardiovascular disorders and/or liver disease including administering to a subject a composition including and combination of any of milk thistle or a constituent thereof, vitamin E, carnitine, caffeine, vitamin B₁₂, and S-adenosylmethionine and in some embodiments, milk thistle or a constituent thereof, vitamin E, carnitine and a physiologically acceptable carrier, wherein the diabetes-related disorder or the liver disease is not fatty liver disease or non-alcoholic fatty liver disease (NAFLD).

In some embodiments, the subject has non-alcoholic fatty liver disease (NAFLD). In other embodiments, the subject is obese. In still other embodiments, the subject is morbidly obese.

According to some embodiments of the present invention, the metabolic syndrome is a condition selected from the group consisting of increased blood pressure, a high blood sugar level, excess both fat around the waist, abnormal cholesterol levels, and combinations thereof. In some embodiments, the diabetes-related disorder is selected from the group consisting of hyperglycemia, hyperinsulinemia, impaired glucose tolerance, impaired fasting glucose, non-alcoholic fatty liver disease, dyslipidemia, hypertriglyceridemia, insulin resistance, and combinations thereof. In particular embodiments, the cardiovascular disease is selected from the group consisting of atherosclerotic disease, dyslipidemia, hypercholesterolemia, decreased HDL levels, hypertension, coronary heart disease, coronary artery disease, and combinations thereof. In some embodiments, the liver disease is inflammation, hepatitis, fibrosis, cirrhosis, non-alcoholic fatty liver disease (NAFLD), steatosis, nonalcoholic steatohepatitis (NASH), liver cancer and combinations thereof.

In particular embodiments, the invention relates to methods of preventing or treating metabolic syndrome or metabolic disorders including administering to a subject a composition including milk thistle or a constituent thereof, vitamin E, carnitine, caffeine, vitamin B₁₂, and S-adenosylmethionine, and in some embodiments, milk thistle or a constituent thereof, vitamin E, carnitine, and a physiologically acceptable carrier.

In some embodiments, the methods comprise, consist or consist essentially of the steps recited therein.

Some aspects of the present invention are described in more detail in the following non-limiting Examples. These are not intended to restrict the present invention, and may be modified within the range not deviating from the scope of this invention.

### EXAMPLES

### 1. Objectives of the Study.

### A. Primary Objectives

To determine the effects of Vitamin E, silymarin and carnitine (VSC) on normalization of abnormalities in hepatic function testing.

To determine the effects of Vitamin E, silymarin, carnitine (VSC), caffeine and optionally Vitamin B₁₂ and/or S-adenosylmethionine (SAMe) on normalization of abnormalities in hepatic function testing.

### B. Secondary Objectives

To determine possible improvements in blood glucose and hemoglobin AlC (markers for diabetes), cholesterol, LDL, triglycerides (markers for cardiovascular disease) and CRP a marker of inflammation.

### C. Additional Objectives

To determine the effects of each composition presented herein on preventing or treating metabolic syndrome, metabolic disorders, a diabetes-related disorder, a cardiovascular disorder or liver disease in various subject populations.

### 2. Overview of Study Design and Dosing Regimen.

Patients with either radiographic or histologic diagnosis of NAFLD are randomized to receive either VSC or matching placebo for 24 weeks.

The following labs are measured before and after administration of drug and placebo and at 6 week intervals for up to 6 months: (AST, ALT, triglycerides, HDL, LDL, insulin levels, glucose and CRP).

### 3. Exemplary Formulations

Exemplary formulation 1

| | | |
|---|---|---|
| silymarin | 400 | mg |
| carnitine | 1 | mg |
| Vitamin E | 200 | IU |

Exemplary formulation 2

| | | |
|---|---|---|
| silymarin | 400 | mg |
| carnitine | 1 | mg |
| Vitamin E | 200 | IU |
| Caffeine | 20 | mg |

Exemplary formulation 3

| | | |
|---|---|---|
| silymarin | 400 | mg |
| carnitine | 1 | mg |
| Vitamin E | 200 | IU |
| Vitamin B₁₂ | 2 | mcg |

Exemplary formulation 4

| | | |
|---|---|---|
| silymarin | 400 | mg |
| carnitine | 1 | mg |
| Vitamin E | 200 | IU |
| Caffeine | 20 | mg |
| Vitamin B₁₂ | 2 | mcg |

Exemplary formulation 5

| | | |
|---|---|---|
| silymarin | 400 | mg |
| carnitine | 1 | mg |
| Vitamin E | 200 | IU |
| S-adenosylmethionine | 400 | mg |

Exemplary formulation 6

| | | |
|---|---|---|
| silymarin | 400 | mg |
| carnitine | 1 | mg |
| Vitamin E | 200 | IU |
| Caffeine | 20 | mg |
| S-adenosylmethionine | 400 | mg |

Exemplary formulation 7

| | | |
|---|---|---|
| silymarin | 400 | mg |
| carnitine | 1 | mg |
| Vitamin E | 200 | IU |
| Vitamin B₁₂ | 2 | mcg |
| S-adenosylmethionine | 400 | mg |

Exemplary formulation 8

| | | |
|---|---|---|
| silymarin | 400 | mg |
| carnitine | 1 | mg |
| Vitamin E | 200 | IU |
| Caffeine | 20 | mg |
| Vitamin B₁₂ | 2 | mcg |
| S-adenosylmethionine | 400 | mg |

The amount of each individual component in the composition is between about 0.1-4000 mg (including all numerical values in between).

The foregoing is illustrative of the present invention, and is not to be construed as limiting thereof. The invention is defined by the following claims, with equivalents of the claims to be included therein.

That which is claimed is:

1. A composition comprising:
(i) milk thistle or a constituent thereof;
(ii) vitamin E;
(iii) carnitine;
(iv) caffeine;
(v) vitamin B₁₂; and
(vi) a physiologically acceptable carrier.

2. The composition of claim 1 further comprising S-adenosylmethionine (SAMe).

3. The composition of claim 1, wherein the constituent of milk thistle is selected from the group consisting of silymarin, silybin, silychristin, silydianin isosilychristin, isosilybin, silandrin; silyhermin A, silyhermin B, 2,3 dehydrosilybin, silybinomers, silybin A, silybin B, and combinations thereof.

4. A method of preventing or treating metabolic syndrome, a diabetes-related disorder, a cardiovascular disorder or liver disease comprising administering to a subject a composition of claim 1.

5. A method of preventing or treating metabolic syndrome, a diabetes-related disorder, a cardiovascular disorder or liver disease comprising administering to a subject a composition comprising milk thistle or a constituent thereof, vitamin E, carnitine and a physiologically acceptable carrier, wherein the diabetes-related disorder or the liver disease is not fatty liver disease or non-alcoholic fatty liver disease (NAFLD).

6. The method of claim 5, wherein the subject is obese.

7. The method of claim 5, wherein the subject is morbidly obese.

8. The method of claim 5, wherein the metabolic syndrome is a condition selected from the group consisting of increased blood pressure, a high blood sugar level, excess body fat around the waist, abnormal cholesterol levels, and combinations thereof.

9. The method of claim 5, wherein the diabetes-related disorder is selected from the group consisting of hyperglycemia, hyperinsulinemia, impaired glucose tolerance, impaired fasting glucose, non-alcoholic fatty liver disease, dyslipidemia, hypertriglyceridemia, insulin resistance, and combinations thereof.

10. The method of claim 5, wherein the cardiovascular disease is selected from the group consisting of atherosclerotic disease, dyslipidemia, hypercholesterolemia, decreased HDL levels, hypertension, coronary heart disease, coronary artery disease, and combinations thereof.

11. The method of claim 4, wherein the liver disease is inflammation, hepatitis, fibrosis, cirrhosis, non-alcoholic fatty liver disease (NAFLD), steatosis, nonalcoholic steatohepatitis (NASH), liver cancer and combinations thereof.

12. The method of claim 5, wherein the liver disease is inflammation, hepatitis, fibrosis, cirrhosis, steatosis, nonalcoholic steatohepatitis (NASH), liver cancer and combinations thereof.
